(19) United States
(12) Patent Application Publication
(10) Pub. No.: US 2026/0249053 A1
(43) Pub. Date: Aug. 27, 2026

(54) HANDHELD CONTROL DEVICE FOR MANIPULATING A STEERABLE GUIDE WIRE

(71) Applicant: Demcon Management & Support b.v., Enschede (NL)

(72) Inventors: Petrus Antonius BESSELINK, Enschede (NL); Adrianus Anton VAN DER STELT, Enschede (NL); Jonathan Johannes ZANDMAN, Enschede (NL)

(73) Assignee: Demcon Management & Support b.v., Enschede (NL)

(21) Appl. No.: 18/995,422

(22) PCT Filed: Jul. 24, 2023
(86) PCT No.: PCT/NL2023/050396

§ 371 Date: Jan. 16, 2025

(30) Foreign Application Priority Data
Jul. 25, 2022 (NL) 2032588

(51) Int. Cl. A61M 25/09

(52) U.S. Cl. CPC A61M 25/09; A61M 2025/09116

(57) ABSTRACT

A handheld control device for manipulating a steerable guide wire for insertion into an interior within a patient body. The guide wire includes two, three or more elongate members arranged for controlling the guide wire upon relative longitudinal movement between the members. The control device includes two, three or more cooperating holders disposed along a longitudinal axis and arranged for holding the guide wire aligned with the longitudinal axis. Each holder is configured for engaging a respective elongate member of the guide wire. The control device further includes an actuating system configured to mutually move at least two of the holders along the longitudinal axis upon manual operation of the actuating system.

The present invention relates to a handheld control device for manipulating a steerable guide wire for insertion into an interior within a patient body. The present invention further relates to a method for manipulating such a steerable guide wire.

A guide wire is a wire used for navigating through narrow spaces within the body of a patient, for example through lumina of a vascular system. The guide wire may for instance assist in positioning a catheter by providing guidance to the desired location. Other applications of guide wires include cardiology, gastroenterology, neurology, and oncology. Guide wires generally comprise a core, a tip, a body, and a coating. Many of the guide wires that are available on the market are limited to one degree of freedom, specifically axial rotation. Often, the tip at a distal end of the guide wire is bent prior to insertion thereof into the patient body. When inserted, the distal end can be rotated by axially rotating the proximal end due to the ability of the guide wire to transmit torque therethrough, also referred to as torquability.

Manipulating guide wires (i.e., steering, controlling, orientating and/or directing the guide wire, in particular the tip thereof) is challenging, especially when the guide wire is to be operated by an operator, such as a surgeon, using only one hand.

It is an object of the present invention, next to other objects, to provide a control device for conveniently manipulating a guide wire inserted into an interior within a patient body.

Thereto, a handheld control device according to claim 1 is provided. More specifically, a handheld control device for manipulating a steerable guide wire for insertion into an interior within a patient body, wherein the guide wire comprises two, three or more elongate members arranged for controlling the guide wire upon relative longitudinal movement between the members, is provided, wherein the control device comprises two, three or more cooperating holders disposed along a longitudinal axis and arranged for holding the guide wire aligned with the longitudinal axis, wherein each holder is configured for engaging a respective elongate member of the guide wire, wherein the control device further comprises an actuating system configured to mutually move at least two of the holders along the longitudinal axis upon manual operation of the actuating system.

Such a handheld control device is particularly suitable to manipulate a guide wire having multiple elongate members for controlling the guide wire. European patent publication No. 3 344 323 for instance discloses a steerable guide wire comprising a tubular sheath with helical slots formed therein, such that a change in length of the tubular sheath causes bending of the distal end of the sheath, and an elongate control element that fits within the sheath and is attached thereto to enable an operator to locally change the length of the tubular sheath by imposing a force to the control element. When the operator pulls at the proximal end of the control element while holding the outer tubular sheath in place, the slots close such that bending of the distal guide wire end, while inserted, is enabled.

More in general, such a guide wire may comprise two or more elongate members arranged for controlling the guide wire upon relative longitudinal movement between the members. According to an example of a guide wire comprising a pull wire, an outer tubular sheath, and an inner tubular sheath therebetween, the distal tip can be bent by longitudinally translating the pull wire relative to the inner tube, wherein the (bent) distal tip is rotated by mutually translating the tubes. By enabling the guide wire to rotate only at the distal tip, friction and/or hysteresis in the guide wire can be minimised. In addition, enabling the guide wire to rotate only at the distal tip allows the torquability of the guide wire to be reduced such that a cheaper, thinner and/or more flexible guide wire can be obtained. Moreover, damage to lumen walls can be reduced when moving only the tip of the guide wire while the remainder of the guide wire remains static.

To longitudinally translate the elongate members relative to each other, movement of the elongate members is preferably actuated at the proximal end of the guide wire, such that the distal end can be manipulated while inserted.

For instance, a first holder may be configured to engage a first elongate member of the guide wire (e.g., a pull wire in the guide wire) at a first location along the guide wire, wherein a second holder may be configured to engage a second elongate member of the guide wire (e.g., an outer sheath of the guide wire) at a second location along the guide wire. Upon actuation, the distance between the holders is adjusted, particularly in the longitudinal direction of the guide wire, to effectuate the mutual longitudinal movement of the guide wire members respectively held by the holders.

Since the actuating system is configured to be manually operated, for instance using the thumb or palm in cooperation with another digit of the hand, the operator can intuitively control the mutual movement of the holders and consequently of the elongate members of the guide wire. In other words, the guide wire can be conveniently steered by the operator via the actuating system. Preferably, the control device has a symmetric configuration that enables the control device to be used by both left-handed and right-handed operators. Thereto, it is preferred if the actuating system is symmetrical with respect to the longitudinal axis, such that the control device can be held for operation in alternately a left hand and a right hand.

Preferably, the control device is a purely mechanical controller. More specifically, it is preferred if the actuating system comprises at least one actuating mechanism configured to (mechanically) actuate the movement of the holders upon manual operation of the actuating mechanism. This way, a simple yet effective control device is obtained, which can be made cost-efficiently. Furthermore, a device without electronics can be disposed, recycled or reused more easily. The control device is thus preferably nonelectrical.

Preferably, the actuating mechanism comprises a linear guiding mechanism arranged to linearly guide at least one of the respective holders along the longitudinal axis.

Preferably, at least one actuating mechanism is provided with at least one grip member associated therewith, wherein the grip member is arranged for operatively engaging the actuating mechanism upon engagement of the grip member by a hand of a user, wherein the grip member is fittingly shaped to be ergonomically engaged by a hand of a user. An increased contact surface between the handheld control device and the hand of the user has the advantage that usability of the handheld control device is enhanced. Furthermore, by means of the grip member the control device can be operated irrespective of the size of the (i.e., by each commonly sized hand), preferably by alternately a left hand and a right hand, such that a universal control device for a guide wire is obtained.

Preferably, the control device is configured to be held by a single hand of a user, wherein the actuating system is configured to be manually operated by the single hand. The control device can be manually operated by a human operator and/or a mechanical manipulator such as a robotic hand.

It is preferred if the longitudinal axis, along which the holders are disposed, is substantially straight or rectilinear. Preferably, the holders are arranged for holding the respective elongate member substantially collinear with the longitudinal axis. This way, a compact control device can be obtained which is efficient in use.

Particularly in the example wherein the elongate members of the guide wire include a pull wire and an outer tubular sheath such that the elongate members are arranged collinear in the guide wire, it is preferred that the holders are disposed along a straight axis for holding the guide wire. In the case of such a guide wire, when the holders are disposed along the longitudinal axis, a first holder can hold the pull wire at a location where the pull wire is exposed, and a second holder can hold the outer tubular member. Preferably, the elongate members are exposed at the proximal end of the guide wire, such that the holders of the control device can be connected to the elongate members at the proximal end.

Preferably, the control device comprises, preferably for each holder, at least one fastener for securing at least one of the elongate members of the guide wire to the respective holder. Thereto, the fastener is preferably configured to detachably arrange onto the elongate member and configured to removably connect to the holder. For convenient insertion of the guide wire into the control device, the fastener may be a clip configured to move, upon connecting the clip to the holder, into a clamping state in which the clip is arranged to clamp the elongate member. The holder may be provided with a receptacle for removably receiving the clip in the clamping state.

According to an embodiment wherein the control device comprises three or more cooperating holders, the actuating system preferably comprises a first actuating mechanism and a second actuating mechanism, wherein the first actuating mechanism is configured to move a first of the holders along the longitudinal axis relative to a second of the holders upon manual operation of the first actuating mechanism, wherein the second actuating mechanism is configured to move a third of the holders along the longitudinal axis relative to the second holder upon manual operation of the second actuating mechanism.

According to a preferred embodiment of the control device, at least one actuating mechanism comprises at least one resilient member interconnecting two holders movable by the actuating mechanism. This way, a control device can be obtained which is efficient in use. The resilient member is preferably biased towards a base state of the actuating mechanism, wherein the actuating mechanism is more preferably configured to mutually move the two holders along the longitudinal axis upon manual deflection of the resilient member relative to the base state. By biasing the actuating mechanism, the actuating mechanism can be configured to move the holders towards each other after being actively operated to move the holders apart, or vice versa. For instance, the actuating mechanism may be configured such that deflecting the resilient member moves the holders apart and the holders passively move towards each other when the resilient member is allowed to return to the base state.

Particularly when using one or more resilient members, the forces that are required to operate the actuating system can be pre-set to optimally suit the operator, as further described further below. A control device comprising resilient members allows for a frictionless controller in which the operating forces are only dependent on the configuration and properties of the resilient members. By removing friction points, the force needed to operate the controller can be minimised. An advantage of low operating forces is that the operator can receive haptic feedback from the controller more accurately. As such, no sensors, processors, or other electronics are required to control the guide wire using the handheld control device. Hence, a purely mechanical controller can be obtained.

Preferably, the resilient member comprises a leaf spring interconnecting, or coupled between, the two holders. A leaf spring provides a reliable biasing force, while also allowing easy manipulation by the user. Preferably in the base state, the leaf spring extends laterally outwards with respect to the longitudinal axis as seen from the holders. The leaf spring is more preferably configured to move the two holders apart along the longitudinal axis upon laterally inwards manual deflection of the leaf spring relative to the base state.

It is then further preferred if at least one of the actuating mechanisms comprises a cooperating set of at least two leaf springs interconnecting the two holders on either side of the holders respectively, wherein the set of leaf springs is configured to move the two holders apart along the longitudinal axis upon laterally inwards manual compression of the set of leaf springs. Interconnecting the two holders by a pair of leaf springs, preferably arranged respectively on either side of the holders, is an efficient way to ensure linear guiding of the holders along the longitudinal axis. As an alternative, a resilient member can be arranged in cooperating manner with a bearing surface.

The set of leaf springs may be formed by a pair of leaf springs. It was however found that, with only two leaf springs in the actuating mechanism, mutual translation of the respective holders in a lateral direction traverse to the longitudinal axis might occur. To constrain the additional degree(s) of freedom of the holders, an additional leaf spring may be included in the set of leaf springs. Alternatively, or additionally, at least one parameter of the pair of leaf springs is adjusted, which may be stiffness and/or thickness of the leaf springs, and/or stroke length of the actuating mechanism, as further described further below. The resulting increase in force required to operate the actuating mechanism is then preferably minimised. Furthermore, by adjusting the stroke length, the control device can be adjusted for different guide wires.

According to the embodiment wherein the control device comprises three or more cooperating holders, each of the first and second actuating mechanisms may comprise a resilient member respectively interconnecting the respective two holders, wherein preferably each of the resilient members is biased towards a base state, wherein the respective actuating mechanism is more preferably configured to mutually move the two holders along the longitudinal axis upon manual deflection of the resilient member relative to the base state.

Preferably, the resilient member of the first actuating mechanism differs in at least one parameter from the resilient member of the second actuating mechanism. The at least one parameter for instance comprises at least one of length, thickness, and height, or in general any parameter related to a stiffness of the resilient member.

Additionally, or alternatively, it is preferred if, in the base state of the resilient members, the first holder and the second holder are mutually spaced further than the third holder and the second holder.

By configuring the respective actuating mechanisms differently, operation of the actuating system can be selectively optimised for each of the actuating mechanisms. In general, by predefining the parameters of the one or more actuating mechanisms, the forces that are required to operate the actuating system can be pre-set to optimally suit the operator. For instance, by adjusting the parameters of a resilient member of an actuating mechanism, the resilient member may be configured to provide a lateral stiffness in a lateral direction, traverse to the longitudinal axis, and a longitudinal stiffness in the longitudinal direction that exceeds the lateral stiffness. To further exemplify, the resilient member of a first actuating mechanism may be stiffer than a resilient member of a second actuating mechanism, for instance when it is preferred that the first actuating mechanism is operated upon exertion of a larger force relative to the force for operating the second actuating mechanism. This may be the case when the first actuating mechanism serves to induce smaller movements of the respective holders.

In the aforenoted embodiments, the second actuating mechanism may comprise a resilient member respectively interconnecting the respective two holders. According to a preferred embodiment of the handheld control device, the second actuating mechanism comprises a longitudinal moving mechanism comprising a manually controllable moving block arranged engageable by the hand of a user in which the user holds the control device, preferably the thumb thereof, wherein the longitudinal moving mechanism is configured to move the one holder along the longitudinal axis relative to another of the holders upon longitudinal moving of the manually controllable moving block. Preferably, one of the respective holders is formed by or provided in or on the moving block.

According to a further preferred embodiment of the handheld control device, the actuating system is bidirectionally configured for selectively moving the at least two holders along the longitudinal axis either apart or towards each other. In other words, the actuating system is preferably configured for selective engagement by a hand of a user in either a first engagement mode, in which the actuating system is configured to mutually move the at least two holders apart, or a second engagement mode, in which the actuating system is configured to mutually move the at least two holders towards each other. The actuating system preferably comprises a first engagement portion, arranged to be engaged by a hand of a user to move the at least two holders apart in the first engagement mode, and a second engagement portion, arranged to be engaged by the hand of the user to move the at least two holders towards each other in the second engagement mode.

Preferably, at least one of the actuating mechanisms, preferably the longitudinal moving mechanism, is unbiased. This way, the actuating mechanism is arranged to be actively operated both to move the holders apart and to move the holders towards each other.

According to a further preferred embodiment, the handheld control device is provided with a marking arranged for indicating a relative position of at least one of the holders, along the longitudinal axis. The marking can assist in correctly engaging the holder with the respective elongate member—i.e., at the correct location along the guide wire, as further described further below.

To assist in aligning the guide wire with the holders, for convenient connection therewith, the control device is preferably provided with a groove extending collinearly with the longitudinal axis for receiving the guide wire therein. The groove thereby functions as an indicative slot for correctly receiving the guide wire in the control device. As a further advantage of providing the groove, during operation of the guide wire using the control device, contact between the guide wire and other parts of the control device other than, e.g., the holders and/or fasteners can be prevented.

According to a further preferred embodiment of the handheld control device, at least one of the holders, preferably each, is movable between a receiving state, in which the holder is open sideways for laterally inserting the respective elongate member into the holder, and an engaging state, in which the holder is configured to secure the inserted elongate member relative to the holder. This way, the elongate member does not have to be axially threaded through the holder. As such, connection of the guide wire to the control device is facilitated.

According to a further preferred embodiment of the handheld control device, the holders and the actuating system are arranged substantially in the same plane. Preferably, the control device is substantially flat.

Advantageously, the control device can be packaged in a compact manner and efficiently stored and transported, which minimises the costs associated with the control device. Furthermore, such a control device can be conveniently held and operated by an operator.

According to a preferred embodiment of the handheld control device, the control device is formed monolithically. That is, at least a substantial part of the control device is formed monolithically, for instance by means of additive manufacturing. By forming at least part of the control device monolithically, manufacturing can be optimised. For instance, the actuating mechanisms, in particular any resilient members thereof, may be formed monolithically with part of the holders. Additional components, such as fasteners for securing the elongate members of the guide wire to the holders, may be formed separately from the monolithically formed part of the control device or may be included therein. The additional components, such as the fasteners, may be disposed after use of the control device, whereas the monolithically formed part may be reused.

A further preferred embodiment of the handheld control device comprises a locking mechanism configured for locking a mutual position of at least two of the holders, wherein the locking mechanism is movable from an unlocked state, in which the at least two holders are mutually movable along the longitudinal axis, to a locked state, in which the locking mechanism is configured to block mutual movement of the at least two holders along the longitudinal axis, preferably in a predetermined mutual position of the at least two holders. Preferably, the locking mechanism is configured for selectively locking the at least two holders in any one of a plurality of predetermined mutual positions. The locking mechanism may comprise a protrusion and at least one corresponding notch arranged to receive the protrusion in the locked state, in which the cooperating protrusion-notch pair is arranged to block the mutual movement of the at least two holders along the longitudinal axis. The protrusion and the notch may be respectively a pin and a corresponding hole.

According to a further preferred embodiment of the handheld control device, at least one holder is configured for releasably engaging the respective elongate member of the guide wire. Preferably, each holder is configured for releasably engaging the respective elongate member of the guide wire. This way, as opposed to being fixed to a specific guide wire, the control device can be reused and/or used in combination with a variety of guide wires and the like.

As such, according to a further independent aspect, a handheld control device for manipulating a steerable guide wire for insertion into an interior within a patient body, wherein the guide wire comprises at least two elongate members arranged for controlling the guide wire upon relative longitudinal movement between the members, is provided, wherein the control device comprises at least two cooperating holders disposed along a longitudinal axis and arranged for holding the guide wire aligned with the longitudinal axis, wherein each holder is configured for releasably engaging a respective elongate member of the guide wire, wherein the control device further comprises an actuating system configured to mutually move the at least two holders along the longitudinal axis upon manual operation of the actuating system. Preferably, the handheld control device includes any one or more features of the aforenoted embodiments.

According to yet another aspect, a method for manipulating a steerable guide wire for insertion into an interior within a body is provided, wherein the method comprises:
providing a guide wire comprising at least two elongate members arranged for controlling the guide wire upon relative longitudinal movement between the members;
providing a handheld control device comprising at least two cooperating holders disposed along a longitudinal axis, wherein each holder is configured to engage a respective elongate member of the guide wire, wherein the control device further comprises an actuating system configured to mutually move at least two of the holders along the longitudinal axis upon manual operation of the actuating mechanism;
mutually moving at least two of the holders along the longitudinal axis by manual operation of the actuating system.

Preferably, the method further comprises the step of inserting the guide wire into the interior within the body, wherein it is further preferred if the at least two holders are mutually moved along the longitudinal axis subsequent to insertion of the guide wire into the interior.

The interior may be any narrow space within any type of body. More specifically, the body may be the body of a patient that may be undergoing any type of medical procedure such as cardiological, gastroenterological, neurological, oncological, or endovascular, preferably any type of minimally invasive surgery. For instance, the interior within the patient body may be a blood vessel.

The method, in particular the step of providing the handheld control device, preferably comprises:
inserting the elongate members respectively into the holders;
securing the inserted elongate members respectively relative to the holders.

Preferably, each holder is configured for releasably engaging the respective elongate member of the guide wire.

Furthermore, the guide wire is preferably inserted into the interior within the body at least subsequent to insertion of the elongate members into the holders, preferably subsequent to the step of securing the inserted elongate members relative to the holders.

Prior to the step of securing the inserted elongate members respectively relative to the holder, the method preferably comprises:
determining a relative position of at least one of the holders, along the longitudinal axis, relative to a predetermined position along the respective elongate member of the guide wire;
aligning the at least one holder with the predetermined position along the respective elongate member. This way, it can be ensured that the holder, or each holder, engages the respective elongate member of the guide wire at the correct location along the guide wire. For instance, in the example of the guide wire comprising a pull wire, an outer tubular sheath, and an inner tubular sheath therebetween, it is preferred if the holders can be conveniently positioned along the guide wire such that the inner tubular member can be held at a location where the inner tubular member is exposed, and the pull wire can be held at a location where the pull wire is exposed.

To further assist in correctly securing the inserted elongate members respectively relative to the holder, the control device is preferably provided with a marking arranged for indicating a relative position of the holder(s), along the longitudinal axis.

Preferably, the method further comprises the step of processing the control device for reuse, wherein the processing step preferably comprises the step of cleaning the control device.

According to a further independent aspect, a method for connecting a steerable guide wire, for insertion into an interior within a body, to a handheld control device for manipulating the steerable guide wire is provided, wherein the method comprises:
providing a guide wire comprising at least two elongate members arranged for controlling the guide wire upon relative longitudinal movement between the members;
providing the handheld control device, wherein the handheld control device comprises at least two cooperating holders disposed along a longitudinal axis, wherein each holder is configured for releasably engaging a respective elongate member of the guide wire, wherein the control device further comprises an actuating system configured to mutually move at least two of the holders along the longitudinal axis upon manual operation of the actuating mechanism;
inserting the elongate members respectively into the holders;
securing the inserted elongate members relative to the respective holders.

As such, as opposed to being fixed to a specific guide wire, the control device can be used in combination with a variety of guide wires and the like. Alternatively, or additionally, the control device and/or the guide wire can be reused by releasing the elongate members of the guide wire from the holders of the control device.

It is to be appreciated that the guide wire is preferably connected to the control device prior to insertion of the guide wire into the interior within the body.

The method may further comprise the step of mutually moving at least two of the holders along the longitudinal axis by manual operation of the actuating system.

The actuating system of the handheld control device, provided in the method, may comprise at least one resilient member interconnecting the two holders, wherein the resilient member is biased towards a base state of the actuating system, wherein the actuating system is configured to mutually move the two holders along the longitudinal axis upon manual deflection of the resilient member relative to the base state into a deflected state. According to a preferred embodiment of the method, the at least two holders are mutually moved along the longitudinal axis prior to the step of securing the inserted elongate members respectively relative to the holders, wherein the inserted elongate members are secured relative to the respective holders in the deflected state of the actuating system.

In other words, by selective pretension of the control device when securing the guide wire thereto, the guide wire can be selectively biased. More specifically, when the actuating system is moved back to its base state after securing the guide wire to the control device, the holders are also moved back to their initial position along the longitudinal axis, thereby mutually moving the secured elongate members and thus biasing the guide wire. As such, the guide wire can be pretensioned such that the control device is to be actuated to straighten the guide wire, which may be preferred in specific applications of the guide wire.

Preferably, the handheld control device that is provided in any of the aforenoted methods is any handheld control device as described herein.

The present invention is hereinafter further elucidated with reference to the attached drawings, wherein:

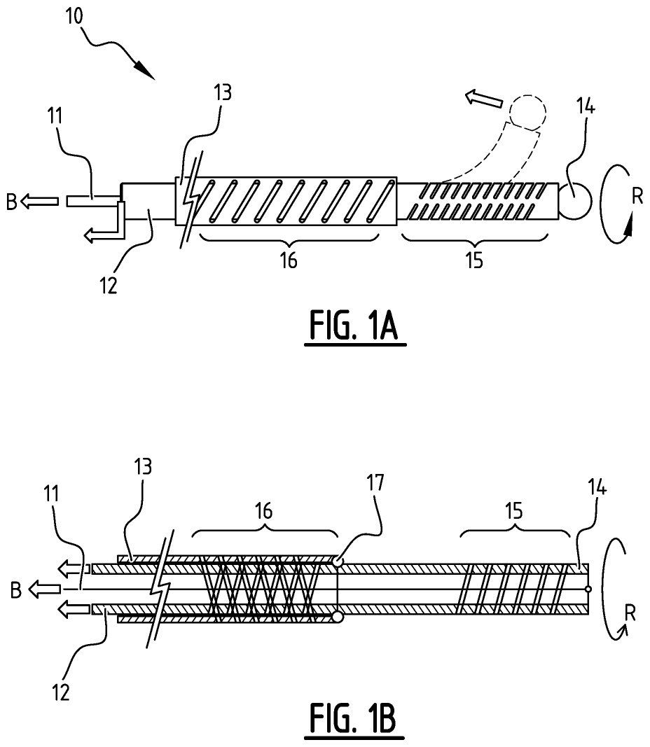

FIG. 1A is a schematic representation of a guide wire;

FIG. 1B depicts a longitudinal section of the guide wire shown in FIG. 1A;

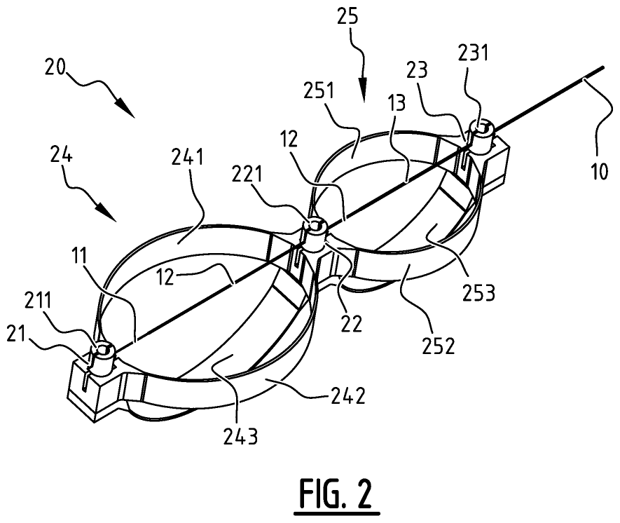

FIG. 2 shows a guide wire controller holding a guide wire as shown in FIGS. 1A-B;

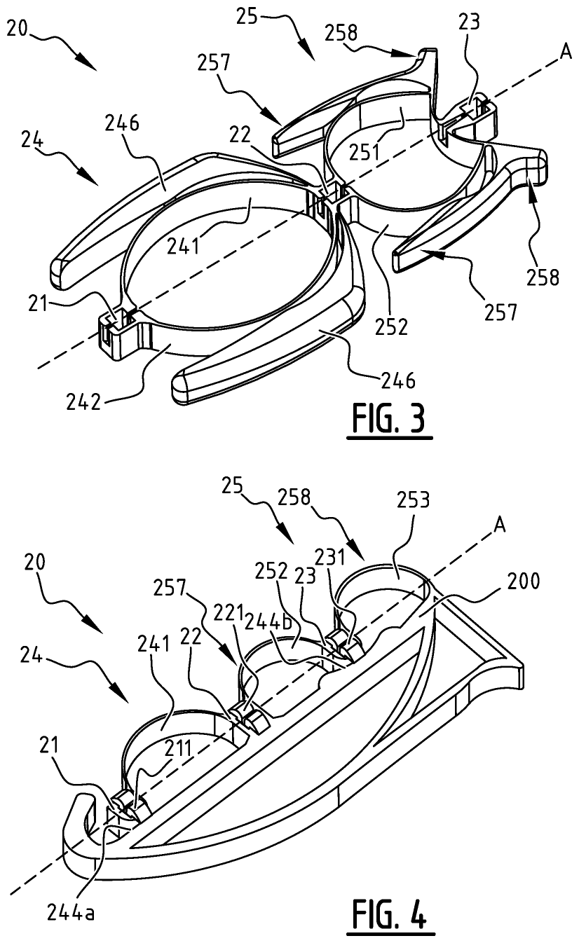

FIGS. 3-7 show various alternative embodiments of the guide wire controller;

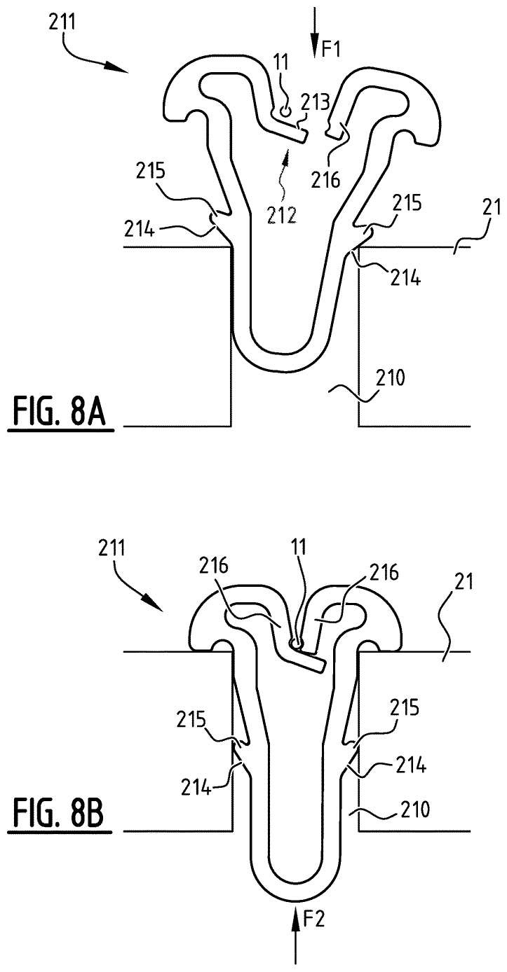

FIGS. 8A-B show a fastener for securing the guide wire to the controller.

In FIGS. 1A-B a guide wire 10 for insertion into an interior within a body, such as the body of a patient undergoing any type of minimally invasive surgery, is schematically represented. The guide wire 10 comprises a pull wire 11, an inner tubular sheath 12 extending collinearly along part of the length of the pull wire 11, and an outer tubular sheath 13 extending collinearly along part of the length of the inner tubular sheath 12. The pull wire 11 and the inner sheath 12 extend from a distal tip 14 of the guide wire 10 toward a proximal end of the guide wire 10, wherein the pull wire 11 extends beyond the inner sheath 12 such that the proximal end of the pull wire 11 is at least partly exposed for imposing a force thereto. The outer sheath 13 extends from near the distal end of the guide wire 10 away from the distal tip 14 such that the distal end of the inner sheath 12 is at least partly exposed. Furthermore, the inner sheath 12 extends towards the proximal end beyond the outer sheath 13 such that the proximal end of the inner sheath 12 is at least partly exposed. The elongate members 11, 12, 13 of the guide wire 10 are exposed at the proximal end of the guide wire 10, such that the guide wire 10 can be operated at the proximal end.

At the distal end, the guide wire 10 has a first section 15 with helical slots formed in the inner tubular sheath 12, wherein adjacent loops of the helix are connected by hinge portions to form a spine-like structure, such that a change in length of the inner sheath 12 causes bending of the distal end of the inner sheath 12. The pull wire 11, extending within the inner sheath 12, is fixed to the inner sheath 12 at the distal tip 14. The distal tip 14 can be moved to a bent position (as illustrated by dashed lines) by axially moving the pull wire 11 relative to the inner sheath 12 in a proximal direction according to a longitudinal translation indicated as B. In other words, when a proximal end of the pull wire 11 is pulled while the inner tubular sheath 12 is held in place, the helical slots in the first section 15 close such that the distal end of the guide wire 10 is bent.

At the distal end of the outer sheath 13, the inner sheath 12 is fixed to the outer sheath 13 at a connection portion 17 and the guide wire 10 has a second section 16 with helical slots formed in both the inner tubular sheath 12 and the outer tubular sheath 13, such that a change in length of the outer sheath 13 causes rotation of the distal end of the inner sheath 12 as indicated by R. The distal tip 14 can be axially rotated by axially moving the inner sheath 12 relative to the outer sheath 13 in a proximal direction. In other words, when a proximal end of the inner sheath 12 is pulled while the outer tubular sheath 13 is held in place, the helical slots in the second section 16 interact such that the distal end of the guide wire 10 is axially rotated. That is, the mechanical interaction between the helical slots of the inner tube 12 and the outer tube 13 in section 16 together can cause the axial rotation of the distal section 15. As such, whether or not the distal tip 14 is bent as described above, the (bent) distal tip 14 can be rotated relative to the proximal end of the guide wire 10 by mutually translating the tubular sheaths 12, 13. Hence, the guide wire 10 is steerable.

In FIG. 2 a guide wire controller 20 is shown. The controller 20 is releasably coupled to the steerable guide wire 10 as depicted in FIGS. 1A-B. It is however noted that the guide wire controller 20 may also be coupled to other guide wires than the type of guide wire shown in FIGS. 1A and 1B. In this example, the controller 20 is coupled to the pull wire 11, the inner sheath 12 and the outer sheath 13 respectively via holders 21, 22, 23. More specifically, a first holder 21 releasably engages the pull wire 11 in the guide wire 10 at a location along the guide wire 10 where the pull wire 11 is exposed, a second holder 22 releasably engages the inner sheath 12 of the guide wire at a location along the guide wire 10 where the inner sheath 12 is exposed, and a third holder 23 releasably engages the outer sheath 13 of the guide wire 10 at a location along the guide wire 10 where the outer sheath 13 is exposed.

The controller 20 comprises a first actuating mechanism 24 and a second actuating mechanism 25, wherein the first actuating mechanism 24 is configured to axially move the first holder 21 relative to the second holder 22 upon manual operation of the first actuating mechanism 24, and wherein the second actuating mechanism 25 is configured to axially move the third holder 23 relative to the second holder 22 upon manual operation of the second actuating mechanism 25. Upon actuation a distance, between the second holder 22 and at least one of the first holder 21 and the third holder 23, is adjusted in the longitudinal direction of the guide wire 10. As such, when coupled to the steerable guide wire 10, the controller 20 can be operated to regulate the relative axial positions of the pull wire 11, the inner sheath 12 and the outer sheath 13 to steer the guide wire 10. The controller 20 is configured to be held by a single hand of a user, wherein the actuating mechanisms 24, 25 are configured to be manually operated by the single hand. More specifically, the second actuating mechanism 25 is configured to be operated by the thumb in cooperation with the index finger, and the first actuating mechanism 24 is configured to be operated by the middle, ring and/or little finger in cooperation with the palm. This allows intuitive operation of the controller 20.

Further reference is made to FIGS. 3-7 showing various alternative embodiments of the guide wire controller 20. The controller 20 comprises three cooperating holders 21, 22, 23 disposed along a straight longitudinal axis A and arranged for holding a guide wire 10 as in FIGS. 1A-2 collinear with the longitudinal axis A. This allows the controller 20 to be conveniently held and operated by for example a surgeon. Each actuating mechanism 24, 25 is configured to mutually move at least two of the holders 21, 22, 23 along the longitudinal axis A upon manual operation of the actuating mechanism 24, 25.

In the embodiments shown in FIGS. 2, 3, 5, 6 and 7, the first actuating mechanism 24 comprises a cooperating pair of leaf springs 241, 242 interconnecting the first holder 21 and the second holder 22 on either side of the holders 21, 22 respectively. The pair of leaf springs 241, 242 is configured to move the holders 21, 22 apart along the longitudinal axis A upon laterally inwards compression of the pair of leaf springs 241, 242. Due to the resilience of the leaf springs 241, 242 the holders 21, 22 move towards each other after being moved apart. In other words, the first actuating mechanism 24 is configured such that compression of the pair of leaf springs 241, 242 moves the holders 21, 22 apart and, when the pair of leaf springs 241, 242 is allowed to return to its base state, the holders 21, 22 passively move towards each other. In the embodiments shown in FIGS. 2-3, the second actuating mechanism 25 comprises, like the first actuating mechanism 24, a cooperating pair of leaf springs 251, 252 interconnecting the second holder 22 and the third holder 23 on either side of the holders 22, 23 respectively. In both the actuating mechanisms 24, 25 of the embodiment shown in FIG. 2, a third leaf spring 243, 253 is added to constrain additional degrees of freedom of the holders 21, 22, 23.

In the embodiment shown in FIG. 4, the first actuating mechanism 24 comprises a first leaf spring 241 singly interconnecting the first holder 21 and the second holder 22 and arranged in cooperating manner with a first bearing surface 244a for linearly guiding the first holder 21. The second actuating mechanism 25 comprises a second leaf spring 252 interconnecting the second holder 22 and the third holder 23, and a third leaf spring 253 connecting the third holder 23 to a frame 200 relative to which the second holder 22 is arranged stationary. The second leaf spring 252 and the third leaf spring 253 are arranged in cooperating manner with a second bearing surface 244b for linearly guiding the third holder 23. The second leaf spring 252 and the third leaf spring 253 enable the second actuating mechanism 25 to be bidirectionally configured such that the second holder 22 and the third holder 23 are either actively moved apart or actively moved towards each other along the longitudinal axis A, as further discussed further below.

In the case of an actuating mechanism (such as the first actuating mechanism 24) comprising a resilient member (such as a leaf spring 241, 242) interconnecting two holders (such as the first holder 21 and the second holder 22), it is possible to secure two of the elongate members of the guide wire 10 (such as the pull wire 11 and the inner sheath 12) to the two holders 21, 22 while the resilient member 241, 242 is deflected from its base state. When the resilient member 241, 242 returns to its base state after securing the elongate members 11, 12 to the holders 21, 22, the holders 21, 22 also return to their initial position along the longitudinal axis A, thereby mutually moving the secured elongate members 11, 12 and thus biasing the guide wire 10. By selective pretension of the controller 20 when securing the guide wire 10 thereto, the guide wire 10 can be selectively biased.

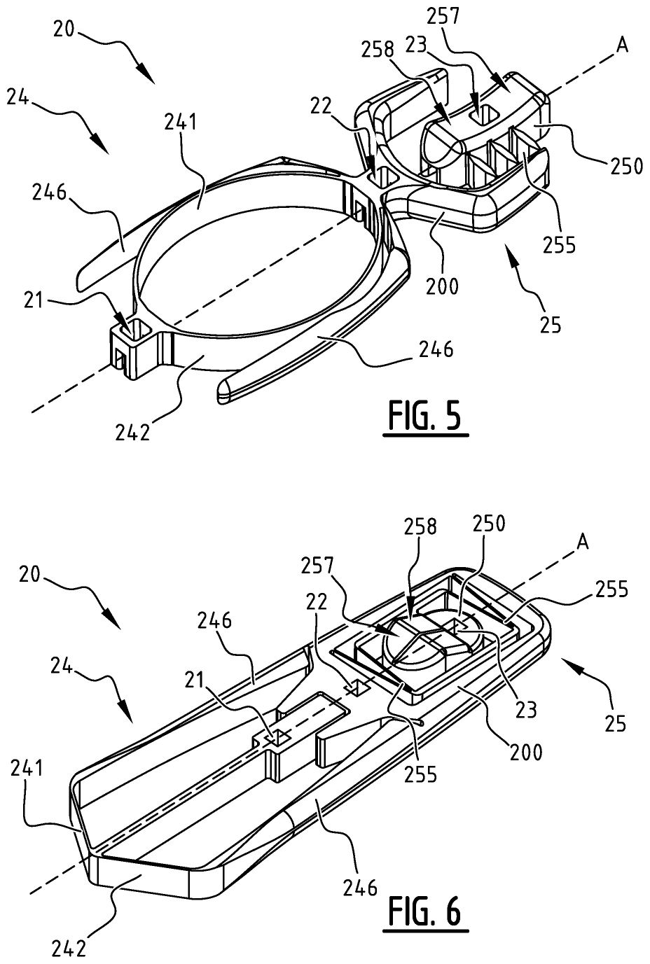

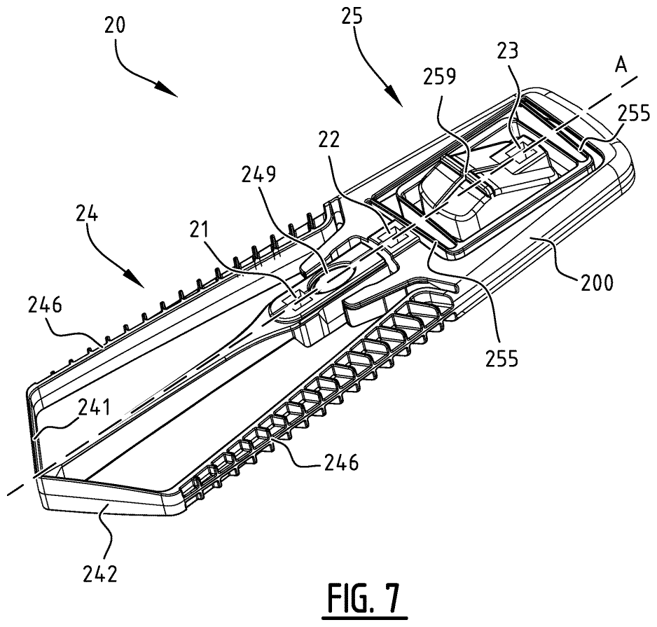

In the embodiments shown in FIGS. 5-7, the second actuating mechanism 25 is a longitudinal moving mechanism comprising a manually controllable moving block 250 arranged engageable by the thumb of the hand of a user in which the user holds the controller 20. The third holder 23 is formed in the moving block 250, such that the longitudinal moving mechanism is configured to move the third holder 23 along the longitudinal axis A towards or away from the second holder 22 upon longitudinal moving of the block 250. The longitudinal moving mechanism further comprises a set of plate-like members 255 connecting the movable block 250 to a frame 200 of the controller 20 and arranged to linearly guide the moving block 250 along the longitudinal axis A. Specifically, in FIG. 5, the plate-like members 255 are arranged in an array, mutually parallel and traverse to the longitudinal axis A, to suppress movement of the block 250 other than a linear movement along the axis A. In FIGS. 6-7, the plate-like members 255 are arranged traverse to the longitudinal axis A and formed by leaf springs, thus allowing some extent of longitudinal movement of the block 250, to form a linear guiding mechanism. As indicated in FIG. 7, the controller 20 is provided with grooves 249, 259 extending collinearly with the longitudinal axis A for receiving a guide wire therein. For instance, a groove 249 is provided adjacent the first holder 21 to prevent detrimental contact between the guide wire and the controller 20 and/or to function as an indicative slot for correctly receiving the respective elongate member of the guide wire in the first holder 21. An additional groove 259 is provided adjacent the third holder 23 for a similar purpose.

Although, in the shown embodiments, linear guidance of the holders 21, 22, 23 along the longitudinal axis A is achieved by resilient members 241, 242, 243, 251, 252, 253, bearing surfaces 244a, 244b and/or other plate-like members 255, it is to be appreciated that various alternative linear guiding mechanisms, or combinations thereof, can be envisaged, such as sliding mechanisms and the like.

In the embodiments shown in FIGS. 3, 5, 6 and 7, each leaf spring 241, 242 is provided with an ergonomically shaped grip member 246 associated therewith and arranged for operatively engaging the respective leaf spring 241, 242 upon engagement of the grip member 246 by a hand of a user for enhanced usability of the controller 20.

In the embodiments shown in FIGS. 3-7, the second actuating mechanism 25 is bidirectionally configured such that the second holder 22 and the third holder 23 are either actively moved apart or actively moved towards each other along the longitudinal axis A. Thereto, the actuating mechanism 25 comprises a first manual engagement portion 257 for moving the second holder 22 and the third holder 23 apart, and a second manual engagement portion 258 for moving the second holder 22 and the third holder 23 towards each other.

The controllers 20 shown in FIGS. 2-7 are each formed monolithically. That is, the actuating mechanisms 24, 25 are formed monolithically with the holders 21, 22, 23. In the embodiments shown in FIGS. 2 and 4, additional fasteners 211, 221, 231 for securing the guide wire 10 to the holders 21, 22, 23 are formed separately from the monolithically formed holders 21, 22, 23 and actuating mechanisms 24, 25.

FIGS. 8A-B show the securing of the pull wire 11 to the first holder 21, by means of a resilient clip 211, in more detail, wherein similar fasteners 221, 231 may be applied for securing the other elongate members 12, 13 of the guide wire 10 to the respective holders 22, 23 in any of the shown embodiments.

First, as shown in FIG. 8A, the clip 211 may be partly received in an unclamped state by a receptacle 210 of the holder 21, formed by an opening, and the wire 11 may be received by a clamping portion 212 of the clip 211. Preferably, the clamping portion 212 is provided with a receiving surface 213 arranged for maintaining the wire 11 at the clamping portion 212 in the unclamped state of the clip 211.

Then, by urging the clip 211 further into the receptacle 210, e.g. by pushing as indicated by F1, the clip 211 is moved into a clamping state in which the clamping portion 212 clamps the wire 11, as shown in FIG. 8B. More specifically, the clip 211 may be provided with a sloping surface 214 arranged to engage an edge of the receptacle 210 to move the clip 211 into the clamping state upon urging the clip 211 into the receptacle. Preferably, the clip 211 comprises outwardly protruding anchor elements 215 arranged to secure the clip 211 in the clamping state. As shown in FIGS. 8A-B, the anchor elements 215 may be provided with the sloping surface 214.

To prevent the clip 211 from clamping the wire 11 with excessive force and thereby damaging the wire 11, the clamping portion 212 preferably comprises at least one resilient element 216 arranged to clamp the wire 11 in the clamping state. The resilient member 216 is arranged to limit or dampen the clamping force exerted on the wire 11 and/or exert a predetermined force on the wire 11.

To release the wire 11, the clip 211 is urged out of the clamping state, e.g. by pushing the clip 211 in the opposite direction F2. Thereto, the receptacle 210 is preferably open towards, in addition, the opposite side such that the clip 211 in the clamped state is accessible from the opposite direction. Furthermore, in the embodiments shown in FIGS. 3-7, the holders 21, 22, 23 and the actuating mechanisms 24, 25 are arranged in the same plane, such that the controller 20 is flat. A controller 20 having at least one flat side has the advantage that the guide wire 10 can be inserted into the controller 20, e.g. using the clips 211, 221, 231 as described above, while the controller 20 is placed flat on e.g. a table.

1. A handheld control device for manipulating a steerable guide wire for insertion into an interior within a patient body, the guide wire comprising at least three elongate members arranged for controlling the guide wire upon relative longitudinal movement between the members, wherein the control device comprises at least three cooperating holders disposed along a longitudinal axis and arranged for holding the guide wire aligned with the longitudinal axis, wherein each holder is configured for engaging a respective elongate member of the guide wire, wherein the control device further comprises an actuating system configured to mutually move at least two of the holders along the longitudinal axis upon manual operation of the actuating system.

2. The handheld control device according to claim 1, wherein the longitudinal axis is substantially straight, wherein each holder is arranged for holding the respective elongate member substantially collinear with the longitudinal axis.

3. The handheld control device according to claim 1, wherein the actuating system comprises a first actuating mechanism and a second actuating mechanism, wherein the first actuating mechanism is configured to move a first of the holders along the longitudinal axis relative to a second of the holders upon manual operation of the first actuating mechanism, wherein the second actuating mechanism is configured to move a third of the holders along the longitudinal axis relative to the second holder upon manual operation of the second actuating mechanism.

4. The handheld control device according to claim 3, wherein at least one of the actuating mechanisms comprises at least one resilient member interconnecting the two holders, wherein the resilient member is biased towards a base state of the actuating mechanism, wherein the actuating mechanism is configured to mutually move the two holders along the longitudinal axis upon manual deflection of the resilient member relative to the base state.

5. The handheld control device according to claim 4, wherein the resilient member comprises a leaf spring interconnecting the two holders, wherein in the base state the leaf spring extends laterally outwards with respect to the longitudinal axis as seen from the holders, wherein the leaf spring is configured to move the two holders apart along the longitudinal axis upon laterally inwards manual deflection of the leaf spring relative to the base state.

6. The handheld control device according to claim 5, wherein at least one of the actuating mechanisms comprises a cooperating set of at least two leaf springs interconnecting the two holders on either side of the holders respectively, wherein the set of leaf springs is configured to move the two holders apart along the longitudinal axis upon laterally inwards manual compression of the set of leaf springs.

7. The handheld control device according to at least claim 3, wherein at least one of the actuating mechanisms is provided with at least one grip member associated therewith, wherein the grip member is arranged for operatively engaging the actuating mechanism upon engagement of the grip member by a hand of a user, wherein the grip member is fittingly shaped to be ergonomically engaged by a hand of a user.

8. The handheld control device according to at least claim 4, wherein each of the first and second actuating mechanisms comprises a resilient member respectively interconnecting the respective two holders, wherein each of the resilient members is biased towards a base state, wherein the respective actuating mechanism is configured to mutually move the two holders along the longitudinal axis upon manual deflection of the resilient member relative to the base state.

9. (canceled)

10. (canceled)

11. (canceled)

12. The handheld control device according to claim 3, wherein the second actuating mechanism comprises a longitudinal moving mechanism comprising a manually controllable moving block arranged engageable by the hand of a user in which the user holds the control device, wherein the longitudinal moving mechanism is configured to move the one holder along the longitudinal axis relative to another of the holders upon longitudinal moving of the manually controllable moving block.

13. The handheld control device according to claim 1, further comprising at least one fastener for securing at least one of the elongate members of the guide wire to the respective holder, wherein the fastener is configured to attach onto the elongate member, wherein the fastener is further configured to connect to the holder.

14. The handheld control device according to claim 1, wherein the actuating system is bidirectionally configured for selectively moving the at least two holders along the longitudinal axis either apart or towards each other, wherein the actuating system is configured for selective engagement by a hand of a user in either a first engagement mode, in which the actuating system is configured to mutually move the at least two holders apart, or a second engagement mode, in which the actuating system is configured to mutually move the at least two holders towards each other.

15. (canceled)

16. The handheld control device according to claim 1, wherein at least one holder is configured for releasably engaging the respective elongate member of the guide wire.

17. The handheld control device according to claim 1, wherein the control device is provided with a marking arranged for indicating a relative position of at least one of the holders, along the longitudinal axis.

18. The handheld control device according to claim 1, wherein at least one of the holders is movable between a receiving state, in which the holder is open sideways for laterally inserting the respective elongate member into the holder, and an engaging state, in which the holder is configured to secure the inserted elongate member relative to the holder.

19. The handheld control device according to claim 1, wherein the holders and the actuating system are arranged substantially in the same plane, wherein the control device is substantially flat.

20. (canceled)

21. (canceled)

22. A method for manipulating a steerable guide wire for insertion into an interior within a body, wherein the method comprises:
providing a guide wire comprising at least two elongate members arranged for controlling the guide wire upon relative longitudinal movement between the members;
providing a handheld control device comprising at least two cooperating holders disposed along a longitudinal axis, wherein each holder is configured to engage a respective elongate member of the guide wire, wherein the control device further comprises an actuating system configured to mutually move at least two of the holders along the longitudinal axis upon manual operation of the actuating mechanism;
mutually moving at least two of the holders along the longitudinal axis by manual operation of the actuating system.

23. The method according to claim 22, further comprising the steps of inserting the elongate members respectively into the holders and securing the inserted elongate members respectively relative to the holders.

24. The method according to claim 23, wherein the actuating system comprises at least one resilient member interconnecting the two holders, wherein the resilient member is biased towards a base state of the actuating system, wherein the actuating system is configured to mutually move the two holders along the longitudinal axis upon manual deflection of the resilient member relative to the base state into a deflected state, wherein the at least two holders are mutually moved along the longitudinal axis prior to the step of securing the inserted elongate members respectively relative to the holders, wherein the inserted elongate members are secured relative to the respective holders in the deflected state of the actuating system.

25. The method according to claim 23, further comprising, prior to the step of securing the inserted elongate members respectively relative to the holder, the steps of determining a relative position of at least one of the holders, along the longitudinal axis, relative to a predetermined position along the respective elongate member of the guide wire and aligning the at least one holder with the predetermined position along the respective elongate member.

26. The method according to claim 22, wherein the handheld control device is a handheld control device according to claim 1.
